(19) United States
(12) Patent Application Publication
(10) Pub. No.: US 2007/0287980 A1
(43) Pub. Date: Dec. 13, 2007

(54) Absorbent article having refastenable and non-refastenable seams

(76) Inventors: Mark James Kline, Okeana, OH (US); Jeromy Thomas Raycheck, Lebanon, OH (US); Mary Lee Amirpour, West Chester, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/810,922

(22) Filed: Jun. 7, 2007

Related U.S. Application Data
(60) Provisional application No. 60/811,614, filed on Jun. 7, 2006.

(51) Int. Cl. A61F 13/15

(52) U.S. Cl. 604/385.24; 604/392; 604/391

(57) ABSTRACT

A disposable absorbent article includes a first side panel, a second side panel, and an intermediate side panel. The first side panel is disposed in a first waist region, and the second side panel is disposed in a second waist region. Both the first and second side panels extend outward from a first longitudinal edge. The first side panel has a first stretchable width, and the second side panel has a second stretchable width. The intermediate side panel is joined to the second side panel thereby forming a non-refastenable seam and is joined to the first side panel thereby forming a refastenable seam. A gap disposed between the first side panel and non-refastenable seam having a gap width between about 1 percent to about 40 percent of the first stretchable width plus the second stretchable width.

### CROSS REFERENCE TO RELATED APPLICATION

This application claims the benefit of U.S. Provisional Application No. 60/811,614, filed Jun. 7, 2007, the substance of which is incorporated herein by reference.

### FIELD OF THE INVENTION

The present invention relates to pull-on diapers. More particularly, the present invention relates to pull-on diapers having a refastenable fastening seam and a non-refastenable fastening seam.

### BACKGROUND OF THE INVENTION

Disposable absorbent articles such as diapers are widely used to absorb exudates by a wearer. In general, the diaper includes elasticity in the leg regions and the waist region of the article. This elasticity typically allows the diaper to expand and contract about the waist and the legs of a wearer. This capability allows the diaper to fit a wide variety of wearers while still providing a good fit of the diaper to the wearer. A type of diaper which has gained much popularity is the pull-on.

Pull-on diapers are generally pre-fastened within the package as opposed to taped diapers which are generally not pre-fastened in a package. As such, pull-on diapers are generally donned on a wearer by pulling on the pull-on diaper similar to underwear. However, because the pull-on diaper is generally donned on the wearer similar to underwear, pull-on diapers present some challenges to diaper designers regarding fit of the pull-on diaper to the wearer.

For example, the starting product waist circumference (no applied tension) should be small enough such that when donned on a smaller wearer, sufficient tension is applied about the waist of the wearer. The tension supplied about the waist of the wearer can be essential in ensuring that the pull-on does not slip down off of the waist of the wearer. In contrast, for larger wearers generally a slightly larger waist circumference may be preferred; however, doing so may preclude a good fit for the smaller wearers. As such, in order to provide a comfortable fit for the larger wearers, the pull-on should be easily extensible such that the waist circumference can be easily expanded in order to fit larger wearers of the pull-on diaper.

Some considerations, in balancing the above competing interests are the overall diaper circumference, the width of the stretch material, and modulus of elasticity of the stretch material. In general, the width of the stretch material is maximized such that smaller waist circumferences can be achieved while also being easily extensible in order to allow the waist circumference to expand such that larger wearers may also be provided with a good fit.

Some pull-ons include refastenable fastening systems and some provide non-refastenable fastening systems. The refastenable fastening systems are typically designed so that the refastenable fastening system can be attached, unattached, and subsequently re-attached effectively without removal of a wearer's clothes, e.g. shoes. In contrast, the non-refastenable fastening systems are typically not capable of being unattached and subsequently re-attached effectively without removal of the wearer's clothes, e.g. shoes.

Regardless of the type of fastening system, many of the components of the fastening system are not elastically extensible. In some cases the fastening system can be configured such that a fastening seam, where two components of the fastening system join or overlap, is located on an outer hip region of a wearer. In general, for pull-on diapers, this area is reserved for stretch materials. As stated previously, the components of the fastening system are generally non-elastically extensible. As such, the existence of the fastening components on the outer hip region of the wearer reduces the amount of stretch material available adjacent to the outer hip region of the wearer. The reduced amount of stretch material can adversely affect the competing interests mentioned above.

Consequently, a need exists for a pull-on diaper which provides sufficient amounts of stretch material such that the competing interests above can be satisfied.

### SUMMARY OF THE INVENTION

One aspect of the present invention pertains to a disposable absorbent article which provides fit for large and small wearers. In some embodiments, a disposable absorbent article for wearing about the lower torso of a wearer may includes a chassis having a topsheet, a backsheet attached to at least a portion of the topsheet, an absorbent core disposed between the topsheet and the backsheet; a first waist region disposed adjacent to a first waist edge, a second waist region disposed adjacent to a second waist edge, and a crotch region disposed between the first waist region and the second waist region; a first longitudinal edge and a second longitudinal edge.

The disposable absorbent article further comprises a first side panel, a second side panel, and an intermediate side panel. The first side panel extends outward from the first longitudinal edge and is disposed in the first waist region. The first side panel comprises a first fastening element and has a first stretchable width.

The second side panel extends outward from the first longitudinal edge and is disposed in the second waist region. The second side panel has a second stretchable width.

The intermediate side panel is joined to the second side panel thereby forming a non-refastenable seam. The intermediate side panel includes a second fastening element capable of engaging the first fastening element thereby forming a refastenable seam. The refastenable seam can be prefastened within a package of disposable absorbent articles. As such, upon removal of an article from the package, the caregiver or wearer can don the article as a pull-on garment without having to form the refastenable seam.

A gap is disposed between the first side panel and the second side panel. The gap has a gap width between about 1 percent to about 40 percent of the first stretchable width plus the second stretchable width.

In other embodiments, the disposable absorbent article may include a disposable pant. The disposable pant for wearing about the lower torso of a wearer, includes a chassis having a topsheet, a backsheet attached to at least a portion of the topsheet, an absorbent core disposed between the topsheet and the backsheet; a first waist region disposed adjacent to a first waist edge, a second waist region disposed adjacent to a second waist edge, and a crotch region disposed between the first waist region and the second waist region; a first longitudinal edge and a second longitudinal edge; and a waist opening and a pair of leg openings.

The disposable pant may further comprise a pair of front side panels, a pair of back side panels, and a pair of intermediate side panels. Each of the pair of front side panels each has a first stretchable width. A first front side panel extends outward from the first longitudinal edge in the front waist region and has an outermost edge, and a second front side panel extends outward from the second longitudinal edge in the front waist region and has an outermost edge.

Each of the pair of back side panels has a second stretchable width. A first back side panel extends outward from the first longitudinal edge in the back waist region and has an outermost edge, and a second back side panel extends outward from the second longitudinal edge in the back waist region and has an outermost edge.

One of the pair of intermediate side panels is joined to the first front side panel and the first back side panel thereby forming a first non-refastenable seam and a first refastenable seam, and one of the pair of intermediate side panels is joined to the second front side panel and the second back side panel thereby forming a second non-refastenable seam and a second refastenable seam. The first and second refastenable seams can be prefastened within a package of disposable absorbent articles. As such, upon removal of an article from the package, the caregiver or wearer can don the article as a pull-on garment without having to form the first and second refastenable seams.

A first gap is disposed between the first front side panel and the first back side panel. The first gap has a first gap width. A second gap is disposed between the second front side panel and the second back side panel. The second gap has a second gap width. The first gap width and the second gap width are each between about 1 percent to about 40 percent of the first stretchable width plus the second stretchable width.

Another aspect of the present invention pertains to a process for making a disposable absorbent article in accordance with the present invention. In some embodiments, a method of producing a disposable pant for wearing about the lower torso of a wearer comprises the steps of joining a front side panel to the chassis in the first waist region; joining a back side panel to the chassis in the second waist region; joining the intermediate side panel to the front side panel, thereby creating a first fastening seam; and joining the intermediate side panel to the back side panel, thereby creating a second fastening seam. In some embodiments, the first fastening seam is refastenable and the second fastening seam is non-refastenable.

### BRIEF DESCRIPTION OF THE DRAWINGS

FIG. 1A is a plan view showing a disposable absorbent article constructed in accordance with the present invention with a portion of the disposable absorbent article cut-away to more clearly show the underlying structure of the disposable absorbent article.

FIG. 1B is an isometric view showing the disposable absorbent article of FIG. 1A in a fastened configuration.

FIG. 2 is an elevation side view showing a front side panel, back side panel, an intermediate side panel from the disposable absorbent article of FIG. 1A.

FIG. 3 is a cross sectional view showing another embodiment of the front side panel, back side panel, and intermediate side panel from the disposable absorbent article of FIG. 1A.

FIG. 4 is an elevation side view showing another embodiment of the front side panel, back side panel, and intermediate side panel, from the disposable absorbent article of FIG. 1A.

FIG. 5 is a schematic view showing a plurality of prefastened pull-on diapers constructed in accordance with the present invention in a package.

FIG. 6A is a side elevation view of a tab member and a slot member joined in a first orientation.

FIG. 6B is a side elevation view of the tab member and slot member of FIG. 6A joined in a second orientation.

FIG. 7A is a side elevation view of a fastening system joined in a first orientation.

FIG. 7B is a side elevation view of the fastening system of FIG. 7A joined in a second configuration.

### DETAILED DESCRIPTION OF THE INVENTION

Definitions:

As used herein, the term “absorbent article” refers to devices that absorb and contain body exudates and, more specifically, refers to devices that are placed against or in proximity to the body of a wearer to absorb and contain the various exudates discharged from the body.

“Body-facing”, “inner-facing”, “outer facing”, and “garment-facing”, refer respectively to the relative location of an element or a surface of an element or group of elements. “Body-facing” and “inner-facing” imply the element or surface is nearer to the wearer during wear than some other element or surface. “Garment-facing” and “outer-facing” imply the element or surface is more remote from the wearer during wear than some other element or surface (i.e., element or surface is proximate to the wearer's garments that may be worn over the disposable absorbent article).

As used herein, the term “diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

The term “disposable” is used herein to describe absorbent articles that generally are not intended to be laundered or otherwise restored or reused as absorbent articles (i.e., they are intended to be discarded after a single use and, preferably, to be recycled, composted or otherwise discarded in an environmentally compatible manner).

As used herein “elastically extensible” refers to characteristics of extensible materials that have the ability to return to approximately their original dimensions after a force that extended the extensible material is removed. Herein, any material or element described as “extensible” may also be “elastically extensible” unless otherwise provided.

As used herein “non-elastically extensible” shall refer to characteristics of materials which upon application of a biasing force, can stretch to an elongated length of at least about 110%, preferably 125% of its relaxed, original length (i.e. can stretch to 10 percent, preferably 25% more than its original length), without rupture or breakage, and upon release of the applied force, shows little recovery, less than about 40%, preferably less than about 20% and more preferably less than about 10% of its elongation.

As used herein, the term “relatively inextensible” shall refer to characteristics of materials which upon application of a tensile load (e.g., a load applied parallel to the lateral axis of the article) of about 4.0 N/cm extends less than about 125%, alternatively less than about 115%, alternatively less than about 105% of its relaxed original length (i.e. can extend about 25%, 15%, or 15% more than its original length under the specified load).

As used herein the term “joined” encompasses configurations whereby an element is directly secured to another element by affixing the element directly to the other element, and configurations whereby an element is indirectly secured to another element by affixing the element to an intermediate member(s) which in turn are affixed to the other element. Additionally configurations where an element is permanently secured to another element or removably secured to another element are included.

The term “longitudinal” refers to a direction running from one waist edge of the article to an opposing waist edge of the article and generally parallel to a line which corresponds to the maximum linear dimension of the article. Directions within ±45° of the longitudinal direction are considered to be “longitudinal”.

The term “lateral” refers to a direction running from one side edge of the article to an opposing side edge of the article and generally at a right angle to the longitudinal direction and in the same plane as the longitudinal direction. Directions within ±45° of the lateral direction are considered to be “lateral”.

The terms “pant”, “training pant”, “closed diaper”, “pre-fastened diaper”, and “pull-on diaper”, as used herein, refer to disposable garments having a waist opening and leg openings designed for infant or adult wearers. A pant can be configured such that the pant has a closed waist and leg openings prior to being donned on the wearer, or the pant can be configured such that the waist is closed and the leg openings formed while on the wearer. A pant may be preformed by any suitable technique including, but not limited to, attaching together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened, or rear waist fastened). Examples of suitable pants are disclosed in U.S. Pat. No. 5,246,433; U.S. Pat. No. 5,569,234; U.S. Pat. No. 6,120,487; U.S. Pat. No. 6,120,489; U.S. Pat. No. 4,940,464; U.S. Pat. No. 5,092,861; U.S. Pat. No. 5,897,545; U.S. Pat. No. 5,957,908; and U.S. Patent Publication No. 2003/0233082 A1.

Description:

In one aspect of the present invention, disposable absorbent articles constructed in accordance with the present invention balance the competing interests stated above and provide improved fit and comfort to the user. Additionally, the pull-on diaper of the present invention provides the convenience of a refastenable fastening system to the consumer.

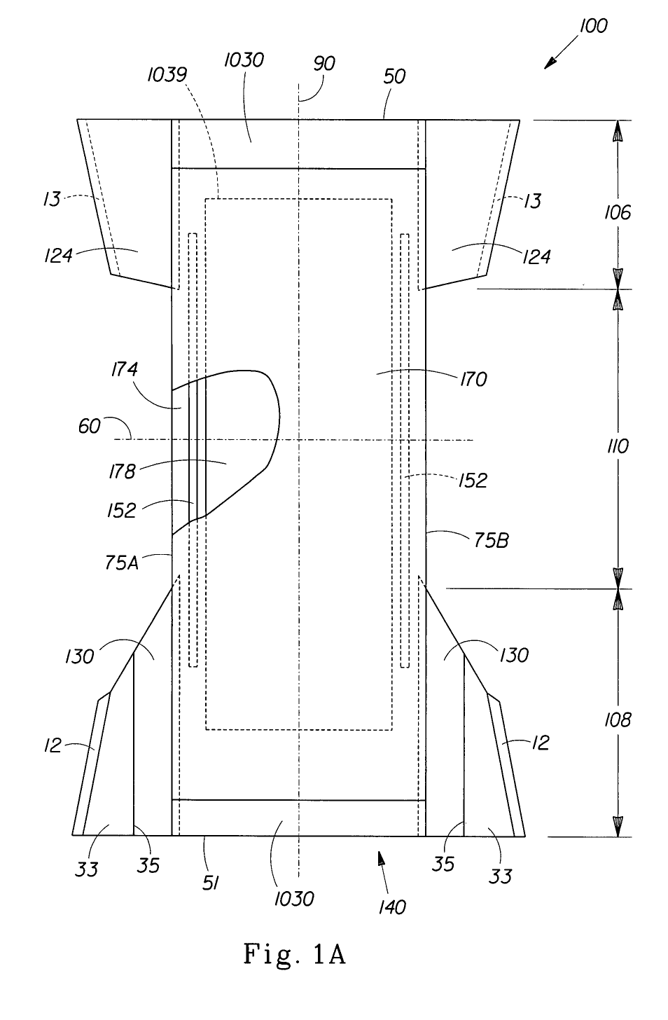

As shown in FIG. 1A, the portion of the disposable absorbent article 100 that faces a wearer is oriented towards the viewer. The disposable absorbent article 100 may comprise a chassis 140 which includes a front waist region 106, a back waist region 108, and a crotch region 110 disposed between the front waist region 106 and the back waist region 108. The chassis 140 may further comprise a topsheet 170, a backsheet 174, and an absorbent core 178. The absorbent core 178 can be positioned between at least a portion of the topsheet 170 and the backsheet 174.

A portion of the periphery of the chassis 140 can be defined by longitudinal edges 75A and 75B; a first waist edge 50, and a second waist edge 51. The longitudinal edges 75A and 75B may run generally parallel to a longitudinal centerline 90 of the disposable absorbent article 100. In some embodiments, for better fit, the longitudinal edges 75A and 75B can be curved or angled to produce an “hourglass” shaped garment when viewed in a plan view. The first waist edge 50 and the second waist edge 51 may run generally parallel to a lateral centerline 60 of the disposable absorbent article 100. The chassis 140 may further comprise elastic leg features 152 which can be disposed adjacent to the longitudinal edges 75A and 75B.

The disposable absorbent article 100 may further comprise a waist feature 1030 which can help provide improved fit and containment of the disposable absorbent article 100 about a wearer. The waist feature 1030 is a portion or zone of the disposable absorbent article 100 which is intended to elastically expand and contract to dynamically fit the wearer's waist. The elastic waist feature 1030 generally extends longitudinally outward from at least one of the waist edges 1039 of the absorbent core 178 and generally forms at least a portion of the first waist edge 50 of the disposable absorbent article 100.

In some embodiments, the elastic waist feature 1030 or any of its constituent elements can include a separate element affixed to the disposable absorbent article 100. In some embodiments, the elastic waist feature 1030 can be constructed as an extension of other elements of the disposable absorbent article 100 such as the backsheet 174, the topsheet 170 or both the backsheet 174 and the topsheet 170. Examples of suitable waist features include those described in U.S. Pat. No. 4,515,595; U.S. Pat. No. 5,151,092; and U.S. Pat. No. 5,221,274. Although disposable diapers are generally constructed so as to have two elastic waist features, as shown in FIG. 1A, one positioned in a first waist region and one positioned in a second waist region, diapers can be constructed with a single elastic waist feature.

The disposable absorbent article 100 may further comprise elasticized leg cuffs 1072 (shown in FIG. 1B) to improve containment of liquids and other body exudates. Each elasticized leg cuffs 1072 (shown in FIG. 1B) may include several different embodiments for reducing the leakage of body exudates in the leg regions. (The leg cuff can also be referred to as leg bands, side flaps, barrier cuffs, or elastic cuffs.) U.S. Pat. No. 3,860,003 describes a disposable diaper which provides a contractible leg opening having a side flap and one or more elastic members to provide an elasticized leg cuff (gasketing cuff). The leg cuffs 1072 may be constructed in any suitable configuration known in the art. Some suitable examples of leg cuff configurations are described in U.S. Pat. No. 4,909,803; U.S. Pat. No. 4,695,278; and U.S. Pat. No. 4,795,454.

Front side panels 124 can extend outboard from the longitudinal edges 75A and 75B of the chassis 140 in the front waist region 106. Back side panels 130 can extend outboard from longitudinal edges 75A and 75B of the chassis 140 in the back waist region 108. Intermediate side panels 33 can be joined to the back side panels 130 and form fastening seams 35. Although not shown, embodiments are contemplated where at least one intermediate side panel 33 is joined to a front side panel 124 thereby forming a fastening seam.

The designation of front and back side panels can be interchangeable and is not meant to restrict the positioning of the specific side panels. For example, the front side panels can be joined to the back waist region and the back side panels can be joined to the front waist region without departing from the spirit of the present invention.

In some embodiments, the front side panels 124 and/or the back side panels 130 can be discrete elements joined to the chassis 140 of the disposable absorbent article 100. In some embodiments, the front side panels 124 and/or the back side panels 130 may comprise a portion of the topsheet 170, the backsheet 174, and/or the leg cuffs 1072 (shown in FIG. 1B). Additionally, suitable combinations are contemplated. For example, in some embodiments, at least one front side panel 124 may comprise a portion of the topsheet 170, the backsheet 174, and/or the leg cuffs 1072, while at least one back side panel 130 are discrete elements joined to the chassis 140 or vice versa.

At least a portion of the front side panels 124 can be elastically extensible. Similarly, at least a portion of the back side panels 130 can be elastically extensible. In contrast, at least a portion of the intermediate side panels 33 is non-elastically extensible and/or relatively inextensible. The non-elastically extensible and/or relatively inextensible intermediate side panels 33 are discussed hereafter.

The fastening seams 35, in some embodiments, can be non-refastenable. For example, the intermediate side panels 33 can be non-refastenably joined to the back side panels 130 by any suitable method known in the art. Some examples of suitable joining methods include a variety of conventional bonding techniques including pressure, thermal, adhesive, ultrasonic bonding, fusion bonding, etc. In some embodiments, the fastening seams 35 can be overlap seams, i.e. an outer-facing surface of the intermediate side panel 33 is joined to an inner-facing surface of the back side panel 130 or vice versa. In some embodiments, the fastening seams 35 may be butt seams, i.e. an inner-facing surface of the intermediate side panel 33 is joined to an inner-facing surface of the back side panel 130. Alternatively, embodiments are contemplated where an outer-facing surface of the intermediate side panel 33 is joined to an outer-facing surface of the back side panel 130. Any suitable combination of overlap and butt seams are contemplated. Additionally, in embodiments where the intermediate side panel 33 is joined (non-refastenably) to the front side panel 124, the configurations discussed above pertaining to the fastening seams 35 are equally applicable. Alternatively, the fastening seams 35, in some embodiments, may be refastenable. In embodiments comprising refastenable fastening seams 35, the fastening seams 35 may be configured as described below with regard to embodiments including refastenable fastening seams 37 (shown in FIG. 1B).

As shown in FIG. 1B, the disposable absorbent article 100 is in the fastened configuration. In the fastened configuration, the disposable absorbent article 100 forms a waist opening 160 and a pair of leg openings 164.

The front side panels 124, back side panels 130, and intermediate side panels 33, of the present invention can form portions of the leg openings 164 when the absorbent article 100, which the side panels are a part of, is fastened. The front side panels 124, back side panels 130, and intermediate side panels 33, form portions of the leg openings 164 which would be disposed on an outer surface of a leg of a wearer. The crotch region 110 (shown in FIG. 1A) of the chassis 140 (shown in FIG. 1A) in conjunction with the first waist region 106 (shown in FIG. 1A) and a second waist region 108 (shown in FIG. 1A) can form portions of the leg openings 164 which would be disposed on an inner surface of the leg of the wearer. Furthermore, both the first waist region 106 (shown in FIG. 1A) and the second waist region 108 (shown in FIG. 1A) can form a portion of the waist opening 160 which would be disposed on a front and rear waist area of the wearer. In contrast, the front side panels 124, back side panels 130, and intermediate side panels 33, can form a portion of the waist opening 164 which would be disposed on a hip area of the wearer.

As shown in FIG. 1B, the front side panels 124 and the intermediate side panels 33 may be joined by fastening elements 13 and 12 thereby forming fastening seams 37. As shown in FIG. 1A, in some embodiments, the fastening elements 12 and 13 are disposed on the intermediate side panels 33 and the front side panels 124, respectively.

In some embodiments, fastening seams 37 can be refastenable. In some embodiments, the fastening elements 12 and 13 may comprise any refastenable fastening elements known in the art. For example, the fastening elements may comprise hook and loop fasteners, macrofasteners, tape fasteners, adhesive fasteners, cohesive fasteners, magnetic fasteners, hermaphroditic fasteners, buttons, snaps, tab and slot fasteners, and the like. Some suitable examples of fastening systems and/or fastening elements are discussed in U.S. Pat. Nos. 3,848,594; 4,662,875; 4,846,815; 4,894,060; 4,946,527; 5,151,092; 5,221,274; 6,432,098; U.S. Pat. No. 6,936,039, U.S. Application Publication No. 2003/0233082 A1; U.S. patent application Ser. No. 11/240,943, entitled, “Anti-Pop Open Macrofasteners” filed on Sep. 30, 2005; U.S. patent application Ser. No. 11/240,838, entitled, “A Fastening System Having Multiple Engagement Orientations”, filed on Sep. 30, 2005.

As shown, in some embodiments, an inner-facing surface of the intermediate side panels 33 can be joined to an outer-facing surface of the front side panels 124. Alternatively, in some embodiments, an inner-facing surface of the front side panels 124 can be joined to an outer-facing surface of the intermediate side panels 33.

Additionally, embodiments, are contemplated where the intermediate side panels 33 are non-refastenably joined to the front side panels 124. In such embodiments, the intermediate side panels 33 can be joined to the front side panels 124 as described above with regard to joining the intermediate side panels 33 to the back side panels 130.

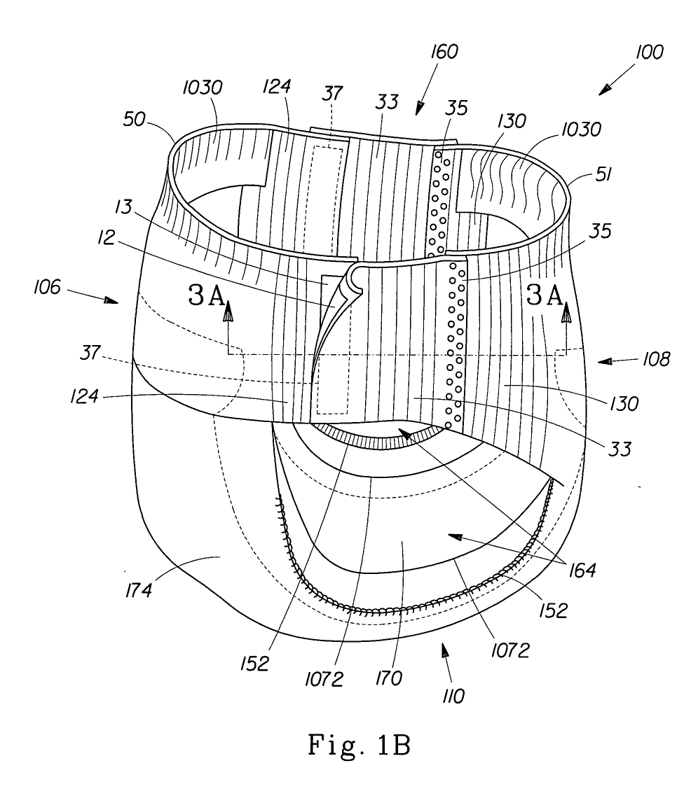

As shown in FIG. 2, the front side panel 124 may comprise a first stretchable width 210. The back side panel 130 has a second stretchable width 220. In some embodiments, although not shown, the front side panel 124 and the back side panel 130 may comprise corrugations when in a relaxed state.

The front side panel 124 may comprise an elastomeric material as described hereafter. The elastomeric material may be the full width of the front side panel 124 or a portion of the width of the front side panel 124. In either case, the first stretchable width 210 is the maximum linear distance generally perpendicular to the longitudinal axis 90 of the disposable absorbent article 100 between the innermost edge 275A of the elastomeric material of the front side panel 124 and an outermost edge 270 of the front side panel 124 when the front side panel 124, back side panel 130, and intermediate side panel 33, are in a relaxed state. The innermost edge 275A of the elastomeric material of the front side panel 124 is the edge of the elastomeric material which is closest to the longitudinal centerline 90 of the article 100 (shown in FIG. 1A). If the location of innermost edge 275A varies along the length of the elastomeric material, the location of the innermost edge 275A is determined by finding the average location relative to the longitudinal centerline 90 of the article 100 (shown in FIG. 1A). In some embodiments, the innermost edge 275A of the elastomeric material may coincide with an innermost edge 270 of the first side panel 124.

The outermost edge 270 of the front side panel 124 is the edge of the front side panel 124 which is furthest from the longitudinal centerline 90 of the article 100 (shown in FIG. 1A). If the location of outermost edge 270 varies along the length of the front side panel 124, the location of the outermost edge 270 is determined by finding the location at which the outermost edge 270 is laterally closest to edge 280B of fastening seam 35.

Similarly, the back side panel 130 comprises an elastomeric material as described hereafter. The elastomeric material may be the full width of the back side panel 130 or a portion of the width of the back side panel 130. The second stretchable width 220 is the maximum linear distance generally perpendicular to the longitudinal axis 90 of the disposable absorbent article between the innermost edge 285A of the elastomeric material of the back side panel 130 and an innermost edge 280A of the fastening seam 35 when the front side panel 124, back side panel 130, and intermediate side panel 33, are in a relaxed state. The innermost edge 285A of the elastomeric material of the back side panel 130 includes the edge of the elastomeric material which is closest to the longitudinal centerline 90 of the article (shown in FIG. 1A). If the location of the innermost edge 285A varies along the length of the elastomeric material, the location of the innermost edge 285A is determined by finding the average location relative to the longitudinal centerline 90 of the article. The innermost edge 280A of fastening seam 35 is the edge of the fastening seam 35 which is closest to the innermost edge 285A of the elastomeric material of the back side panel 130. If the location of innermost edge 280A varies along the length of the back side panel 130, the location of the innermost edge 280A is determined by finding the average location relative to the longitudinal centerline 90 of the article. In some embodiments, the innermost edge 285A of the elastomeric material of the back side panel 130 may coincide with an innermost edge 285 of the back side panel 130.

The first stretchable width 210 of the front side panel 124 does not imply that the entire width of the front side panel 124 is elastically extensible. Instead, at least a portion of the front side panel 124 within the stretchable width 210 of the front side panel 124 is elastically extensible. As an example, the first stretchable width 210 may comprise a portion of the intermediate side panel 33 which may be non-elastically extensible and/or relatively inextensible, in some embodiments.

Similarly, the second stretchable width 220 of the back side panel 130 does not imply that the entire back side panel 130 is elastically extensible. Instead, at least a portion of the back side panel 130 within the second stretchable width 220 is elastically extensible. For example, the second stretchable width 220 may comprise a portion of the intermediate side panel 33 which may be non-elastically extensible and/or relatively inextensible, in some embodiments.

In general, the fastening seams 35 and 37 are non-elastically extensible and/or relatively inextensible regardless of whether they are refastenable or non-refastenable. For example, non-refastenable seams are typically non-elastically extensible. Similarly, refastenable seams generally comprise at least one fastening element which is non-elastically extensible and/or relatively inextensible. For example, in a hook and loop fastening system, the loop element may be elastically extensible; however, the hook material typically is not. As another example, in a tab and slot fastening system, generally neither the tab nor the slot is elastically extensible.

A gap 235 can be defined by the outermost edge 270 of the front side panel 124 and the edge 280B of fastening seam 35 which is laterally closest to outermost edge 270 of front side panel 124 when the front side panel 124, back side panel 130, and intermediate side panel 33, are in a relaxed state. The gap 235 can have a gap width 230 which is the linear distance between the outermost edge 270 of the front side panel 124 and the edge 280B of fastening seam 35. If the gap width 230 varies (i.e., is larger at one longitudinal location than another), the distance which is the smallest, regardless of longitudinal location, is defined as the gap width 230. As stated previously, the intermediate side panel 33, for at least the gap width 230, can be non-elastically extensible and/or relatively inextensible.

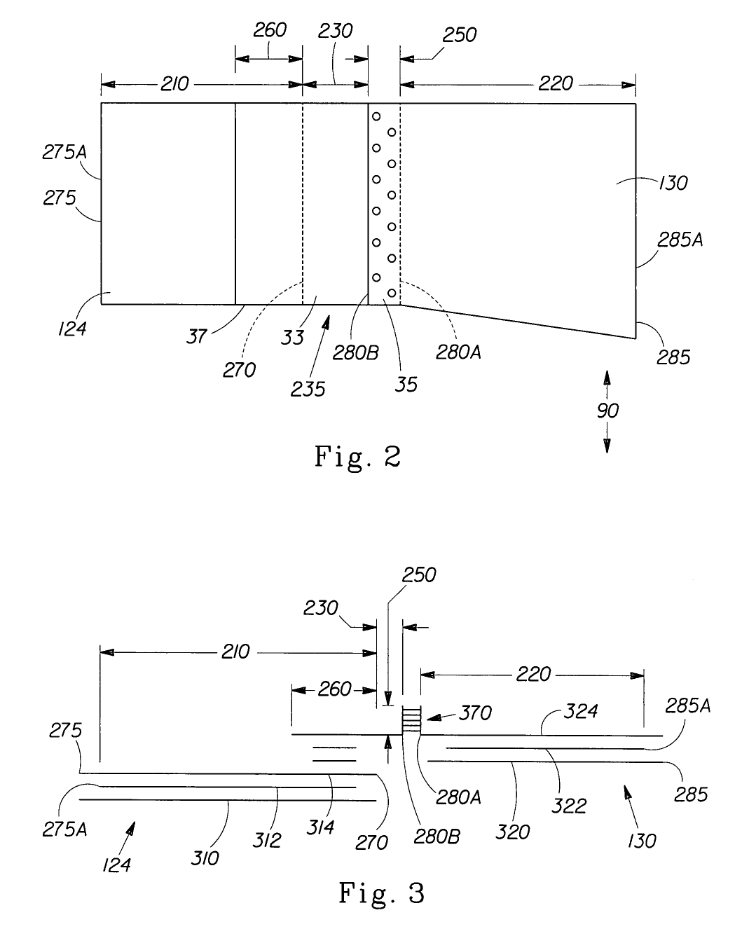

As shown in FIG. 3 and as stated previously, the non-refastenable seam of the present invention may comprise a butt seam 370. As shown, the descriptions provided above regarding the innermost edge 275A of the first side panel 124 and the innermost edge 285A of the second side panel 130 are equally applicable where the non-refastenable seam is a butt seam 370. As shown, in FIG. 3, the butt seam 370 is exaggerated, e.g. the distance between the two elements being joined is shown with a gap in between for ease of illustration. Additionally, regardless of whether the seam 35 is an overlap seam or a butt seam 370, the innermost edge 280A and the edge 280B of the fastening seam 35 can be found as described above.

The gap width 230 can be an important factor in achieving the fit of the product to smaller wearers, e.g. smaller waist circumference, and the larger wearer, e.g. sufficient available stretch material to allow the waist circumference to easily expand. For example, there is a limited amount of distance around the waist of a pant product to fit in all the needed features, including absorbent cores, cuffs, stretch materials, and fasteners placed in regions where they will be adjacent the hip region of the wearer. For example, in portions of the product adjacent the hip region, space may need to be allocated for the first and second stretchable widths 210 and 220, the fastening seam widths 250 and 260, and the gap width 230. All impact the product's fit range and fastening seam performance.

If a gap width 230 is too small, problems between a non-refastenable seam and a refastenable seam may result. For example, a smaller gap width 230 equates to a shorter distance between the non-refastenable seam and the refastenable seam. If no gap is provided, then the refastenable seam may become bonded to the non-refastenable seam during processing. This can reduce or eliminate the refastenability of the refastenable seam. If a small gap is provided, process variations may result in a significant portion of production having the refastenable seam bonded to the non-refastenable seam.

In contrast, a gap width 230 which is too large may require reductions in stretchable width 210, stretchable width 220, and/or fastening seams 35 and 37 to provide a product which fits small wearers well. However, reductions in stretchable widths 210 and 220 limit the fit range of the product (e.g., inability to fit the largest wearers well).

Additionally, reductions in widths of fastening seams 35 and 37 can reduce the strength of the fastening seams. For example, a non-refastenable fastening seam, for example, fastening seam 35, can have a width 250 which is greater than about 5 mm. In some embodiments, the width 250 can be between about 5 mm to about 25 mm, preferably from about 7 mm to about 18 mm or any individual number within the range.

A smaller width 250 for the non-refastenable seam could decrease the peel strength and shear strength of the non-refastenable seam. Similarly, a refastenable seam, for example, fastening seam 37, can have a width 260 between about 5 mm to about 25 mm, preferably from about 7 mm to about 18 mm or any individual number within the range. A smaller width 260 for the refastenable seam could similarly decrease the peel strength and shear strength of the refastenable seam. Further, if a large gap width 230 is used and no reductions to stretchable width 210, stretchable width 220, and/or fastening seams 35 and 37 are made, the product may not fit smaller wearers well.

The size of the gap width 230 is discussed hereafter with regard to the first stretchable width 210 and the second stretchable width 220, cumulatively and separately.

As such, in some embodiments, the gap width 230 can be between about 1% to about 40% of the first stretchable width 210 plus the second stretchable width 220. In some embodiments, the gap width 230 can be between about 3% to about 20% of the first stretchable width 210 plus the second stretchable width 220. In some embodiments, the gap width 230 can be between about 5% to about 10% of the first stretchable width 210 plus the second stretchable width 220.

In some embodiments, the gap width 230 may be less than about 60% of the first stretchable width 210. In some embodiments, the gap width 230 may be less than about 45% of the first stretchable width 210. In some embodiments, the gap width 230 may be less than about 30% of the first stretchable width 210.

In some embodiments, the gap width 230 may be less than about 50% of the second stretchable width 220. In some embodiments, the gap width 230 may be less than about 35% of the second stretchable width 220. In some embodiments, the gap width 230 may be less than about 20% of the second stretchable width 220.

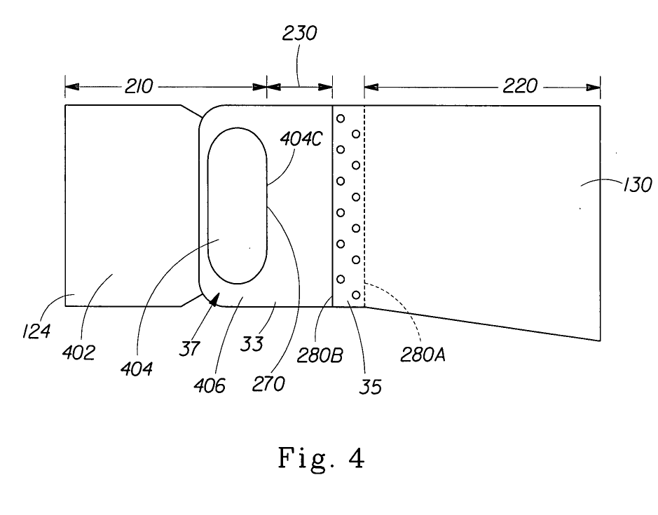

As shown in FIG. 4, in one specific embodiment, the refastenable seam 37 may comprise refastenable fastening elements which include tab and slot fastening members, 402 and 406, respectively. In some embodiments, the first side panel 124 may comprise a tab member 402 having a tab element 404. The second intermediate side panel 33 may comprise a slot member 406. Note that the outermost edge 270 of the front side panel 124 may correspond to an outer edge of the tab element 404. Some examples of suitable configurations for tab and slot fastening system are discussed in U.S. Pat. No. 6,432,098; U.S. Pat. No. 6,936,039, U.S. Application Publication No. 2003/0233082 A1; U.S. patent application Ser. No. 11/240,943, entitled, “Anti-Pop Open Macrofasteners” filed on Sep. 30, 2005; U.S. patent application Ser. No. 11/240,838, entitled, “A Fastening System Having Multiple Engagement Orientations”, filed on Sep. 30, 2005.

In some embodiments, a tab edge 404C may extend to a location which is laterally closer to edge 280B of fastening seam 35 than the outermost edge 270 of first side panel 124. In such embodiments, first stretchable width 210 and gap width 230 are defined relative to edge 404C of tab member 404, with edge 404C replacing outermost edge 270 in the respective definitions.

As discussed above, particularly where the refastenable seams comprise macrofastener elements, the gap width 230 can detrimentally impact the performance of the macrofastener elements. For example, a gap width 230 which is too small, e.g. less than 5 mm can result in a significant number of refastenable fastening elements being non-refastenably joined to the intermediate side panel 33. This can effectively negate the purpose of the refastenable seam.

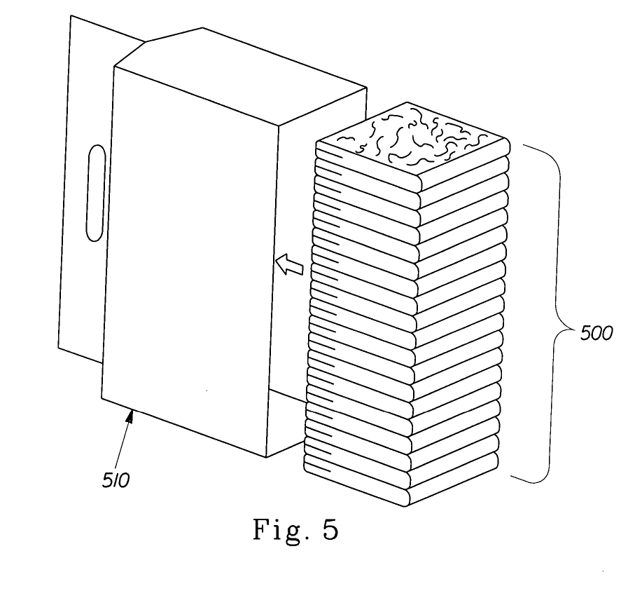

As shown in FIG. 5, embodiments are contemplated where a plurality of disposable absorbent articles 500 constructed in accordance with the present invention are sold in a package 510. The disposable absorbent articles can be prefastened in the package such that the consumer may don an individual article by pulling the article on without having to fasten any fastening elements. Conversely, embodiments are contemplated where at least one disposable absorbent article within the package 510 is sold in an unfastened state. As such, upon removal of the unfastened article from the package, the consumer may have to fasten the article prior to donning the article on the wearer, or the consumer could alternatively apply the article in the conventional manner, e.g. the article is fastened while the article is on the wearer.

In some embodiments, the refastenable seams may be fastened in at least 2 different configurations. For example, the fastening elements of the refastenable seam may be joined in a first fastening configuration which facilitates fastening by a high speed manufacturing line or protects the fasteners for in-use fastening performance. As another example, fastening elements may be joined in a second fastening configuration which is the desired configuration for the end user to form the connection between fastening elements. The first and second fastening configurations are different.

For example, it can be difficult for a high speed manufacturing line to fasten a tab member 404 (shown in FIG. 4) and slot member 406 (shown in FIG. 4). Embodiments are contemplated which can facilitate joining the fastening elements of the refastenable seam in the first orientation.

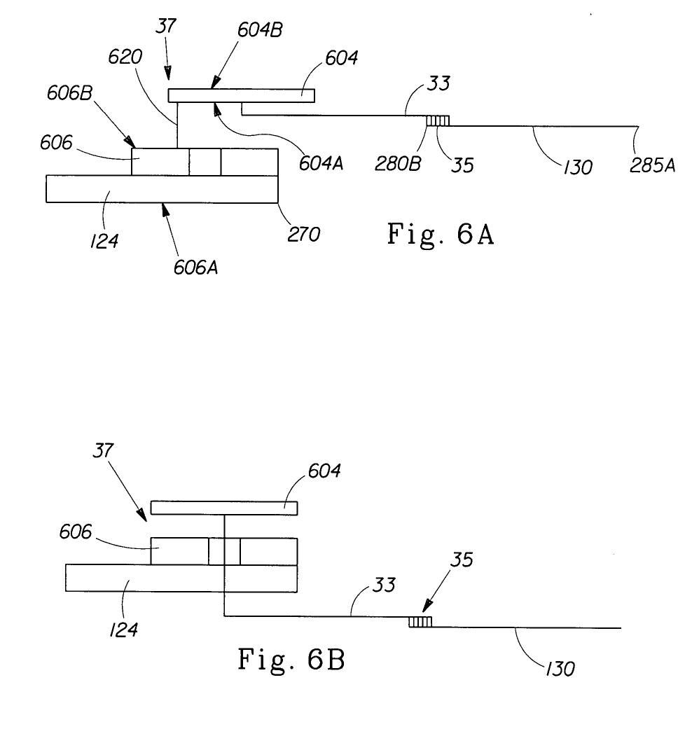

For example, as shown in FIG. 6A, a tab member 604 can be joined to a slot member 606 in the first orientation. As shown, the tab member 604 may be joined to the intermediate side panel 33. Additionally the intermediate side panel 33 may be non-refastenably joined to the back side panel 130 at the fastening seam 35. As stated previously, the non-refastenable fastening seam, e.g. 35, may comprise an overlap seam or a butt seam.

As shown, in some embodiments, the tab member 604 may be joined to the slot member 606 thereby forming the fastening seam 37; however, in a package 510 (shown in FIG. 5), the fastening seam 37 may be configured in the first orientation for the purposes described above. As shown, the first orientation may include joining the tab member 604 to the slot member 606 via at least one first configuration bond 620.

In some embodiments, the first configuration bond 620 may join the tab member 604 with the slot member 606 by bonding a first surface 604A of the tab member 604 and a second surface 606B of the slot member 606. Although not shown, embodiments are contemplated where a second surface 604B of the tab member 604 is joined to a first surface 606A of the slot member 606 in addition to or independently from the first configuration bond 620. Embodiments are contemplated where the first surface 604A and/or the second surface 604B of the tab member 604 are joined to the first surface 606B and/or second surface 606A of the slot member 606.

The first configuration bond 620 between the tab member 404 and the slot member 406 may be refastenable or non-refastenable. In either case, the force to release the first configuration bond should be low enough for a user to easily break the first configuration bond 620 without rendering any parts of the article unsuitable for its intended use. Exemplary refastenable first configuration bonds 620 include any refastenable bonds known in the art, such as certain adhesives, certain cohesives, magnets, hook & loop, and the like. Some suitable examples include an adhesive fastener which joins to a surface coated with a release agent, such as silicone, or a hook which joins to fibrous loop elements, e.g. CS600 hooks and EBL landing zone available from 3M.

Exemplary non-refastenable first configuration bonds 620 include any non-refastenable bonds known in the art, such as certain adhesives, certain cohesives, fusion bonds (i.e., thermal bonds, ultrasonic bonds pressure bonds), and the like. Other suitable examples of non-refastenable first configuration bonds 620 include a light coating of hot melt adhesive, e.g. less than about 20 g/m², preferably less than about 10 g/m², more preferably less than about 5 g/m². Examples of suitable adhesives are available under the designation H2031, H2085, H2988-F02 from Bostik Adhesives.

The first configuration bond 620 may be placed in any suitable location to join portions of adjacent side panels together and may be a single bond or a plurality of bonds. For example, first configuration bond 620 may be in a location which coincides with a fastening element's location or may be adjacent a fastening element. In any case, the user may release the first configuration bond 620, then refasten the tab member 604 and slot member 606 in its second fastening configuration. As shown in FIG. 6B, the tab and slot fastening system of FIG. 6a has been fastened in its second fastening configuration with the tab member 604 having passed through an opening in the slot member 606. As can be seen comparing FIGS. 6A and 6B, the first and second fastening configurations are different.

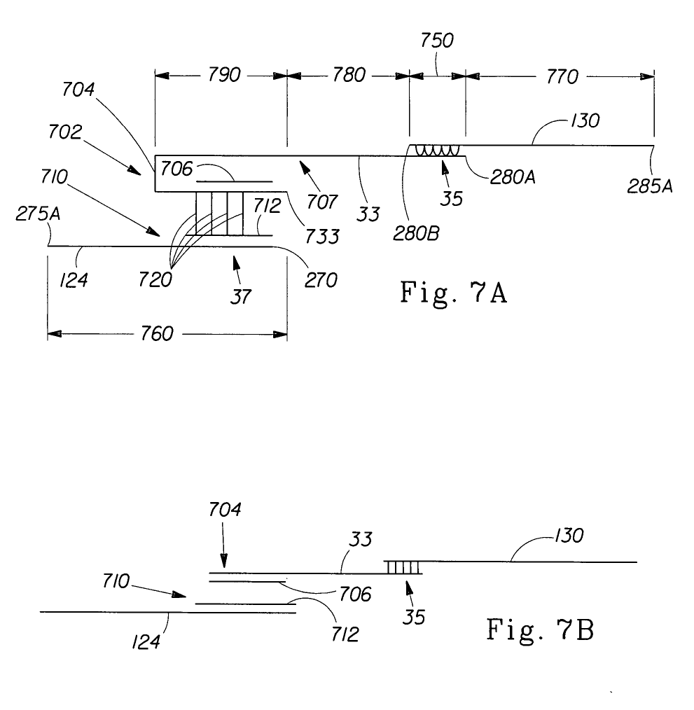

Other fastening systems which are configurable into first and second fastening configurations are contemplated. For example, in some embodiments, as shown in FIG. 7A, a first fastening member 702 may comprise at least one folded over portion 704. The first fastening member 702 may include more than one folded over portion (not shown).

As shown, in some embodiments, the engaging member 702 may be integral with the intermediate side panel 33 or may be discretely joined to the intermediate side panel 33. Additionally the intermediate side panel 33 may be non-refastenably joined to the back side panel 130 at the fastening seam 35. As stated previously, the non-refastenable fastening seam, e.g. 35, may comprise an overlap seam or a butt seam.

As shown, the folded over portion 704 can protect at least a first fastening element 706 from damage until the user unfolds the folded over portion 704. As shown, in some embodiments, the folded over portion 704 may comprise a portion of the intermediate side panel 33. However, in some embodiments, the folded over portion 704 comprise a portion of the first side panel 124, the second side panel 130, and/or the intermediate panel 33.

As shown, the first fastening member 704 can be joined to a second fastening member 710 in a first configuration. In the first configuration, the folded over portion 704 may be joined to a second fastening element 712 via at least one first configuration bonds 720. In some embodiments, the first configuration bond 720 can join the folded over portion 704 to a portion of the first side panel 124. Depending on the type of fastening elements used, an inner surface 707 in contact with the first fastening element 706 may need to be specially configured to assure the first fastening element 706 can release from the inner surface 707 without rendering the first fastening element 706 unusable for its intended purpose (i.e., if the fastening element 706 is an adhesive, the inner surface 707 may be treated with a release coating, as is known in the art). Embodiments are contemplated where the second fastening member 710 comprises a folded portion similar to the folded portion 704 of the first fastening member 702 independently or in conjunction with the folded portion 704 of the first fastening member 702.

As described with regard to FIG. 6A, the force to open first configuration bonds 720 should be low enough to avoid rendering any parts of the article unsuitable for its intended use when the bond is broken, and first configuration bonds may be placed in any suitable location to join adjacent panel. A single or a plurality of first configuration bonds 720 may be used.

FIG. 7B shows the fastening system of FIG. 7A in its second fastening configuration. As shown, in the second configuration, the first fastening element 706 can refastenably join the second fastening element 712. The first and second fastening elements 706 and 712, respectively can be selected from any suitable fastening elements known in the art. For example, the first fastening element and the second fastening elements may be selected from the group consisting of hook and loop, hook and hook, adhesives, cohesives, tab and slot, magnets, and the like.

In most situations for embodiments including at least 2 fastening configurations, definitions of stretchable widths, gap widths, remain consistent with definitions of embodiments previously shown, as do the prefer gap widths as a percent of stretchable widths. In some situations these definitions vary to ensure that no part of fastening seam 37 is bonded into fastening seam 35.

Specifically, as shown in FIG. 7A, first stretchable width 760 is defined differently than previously described herein. In FIG. 7A, the edge 733 of the folded over portion 704 extends to a location laterally closer to outermost edge 280B of fastening seam 35 than the outermost edge 270 of first side panel 124. Therefore, the first stretchable width 760 is defined as the maximum linear distance generally perpendicular to the longitudinal axis 90 (shown in FIG. 1A) of the disposable absorbent article 100 (shown in FIG. 1A) between the innermost edge 275A of the elastomeric material on the first side panel 124 and edge 733 of the folded over portion 704 when the front side panel 124, the intermediate panel 33, and the second side panel 130 are in a relaxed state. Edge 733 of the folded over portion 704 is the edge of the folded over portion 704 which is laterally closest to the edge 280B of fastening seam 35. If the location of edge 733 varies along the length of the folded over portion, the location of edge 733 is determined by finding the location at which edge 733 is laterally closest to edge 280B of fastening seam 35. Then, gap width 780 is defined as the linear distance between edge 733 and edge 280B of fastening seam 35. If gap width 780 varies, the distance which is smallest, regardless of longitudinal location, is defined as gap width 780.

The disposable absorbent articles of the present invention may be constructed in a number of different manners. For example, referring back to FIG. 1A, in some embodiments a front side panel 124 may be joined to the chassis 140 in the first waist region 106, and a back side panel 130 may be joined to the chassis 140 in the second waist region 108. The disposable absorbent article 100 can be folded, and the intermediate side panel 33 can be joined to the front side panel 124 and the back side panel 130. As stated previously, the intermediate side panel 33 can be joined to the front side panel 124 and the back side panel 130 refastenably or non-refastenably.

In general, the order in which the refastenable seam and the non-refastenable seam are created is interchangeable. However, the order in which the refastenable seam and the non-refastenable seam are created can be pertinent in some instances. For example, embodiments including complex fastening elements, e.g. tab and slot, hook and eye, buttons, snaps, etc., the creation of the refastenable seam prior to the non-refastenable seam may facilitate the processing of the disposable absorbent article. Moreover, for complex fastening elements, front side panel 124 may be refastenably joined to the intermediate side panel 33 prior to joining the front side panel 124 to the chassis 140.

The front side panels 124, the back side panels 130, and the intermediate side panels 33 may comprise a variety of materials. For example, in some embodiments, the front side panels 124, the back side panels 130, and/or the intermediate side panels 33, may comprise, in some embodiments, a laminated structure. Specifically, in some embodiments, the front side panels 124, the back side panels 130, and/or the intermediate side panels 33, may comprise a first substrate joined to an elastomeric element. In some embodiments, the front side panels 124, the back side panels 130, and/or the intermediate side panels 33, may further comprise a second substrate joined to the elastomeric element.

An elastomeric element suitable for use in the present invention may include elastic strands or elastic films. Any suitable elastic film known in the art can be used. Some examples of suitable elastic films include polypropylene, polyethylene, polyolefins, styrene-isoprene-styrene, styrene-butadiene-styrene, or combinations thereof. In some embodiments, the basis weight of the elastic films can range from about 10 gsm to about 100 gsm.

Alternatively, or in conjunction with the elastic film, an elastomeric element of the present invention may comprise elastic strands. An example of suitable elastic strands includes those made of a resilient elastic thermoplastic material. The elastic strands may be made from liquid elastic that is extruded through a die to achieve the desired strand elastic diameter and/or shape. Any suitable shape known in the art can be utilized. For example, typical elastic strands have a circular cross sectional shape, but the elastic strands may have different shapes, such as a trilobal shape, or a flat (i.e., “ribbon” like) shape. Some examples of suitable elastic strand shapes include rectangles, circles, ellipses, diamonds, triangles, parallelograms, trapezoids, wedges or other sections of circles or ellipses, other polygons, or other irregular enclosed shapes. Furthermore, the thickness or diameter of the elastic strands may vary in order to accommodate a particular application. For example, in some embodiments, the thickness of elastic strands may be in the range of about 0.02 mm to about 1 mm and the basis weight is in the range of about 20 g/m² to about 300 g/m².

The elastic strands may be applied separately to the substrate, can be extruded onto the substrate, or can be printed onto the substrate. Exemplary apparatuses for applying elastic strands onto a substrate or extruding elastic strands onto a substrate are discussed below. Apparatuses for applying elastic strands in a longitudinal direction are described in U.S. Application Publication No. 2004/0238105 A1 and in U.S. application Ser. No. 10/836,944 entitled “Apparatus for Producing Elastomeric Nonwoven Laminates” filed on Apr. 30, 2004. Apparatuses for applying elastic strands in a transverse direction, an angle from the longitudinal direction, or in a curvilinear fashion are described in U.S. application Ser. No. 10/779,338 entitled “Method of Placing Material Transversely on a Moving Web” filed on Feb. 13, 2004. Apparatuses for applying elastic strands in the longitudinal direction, an angle from the longitudinal direction, or in a curvilinear fashion are described in U.S. application Ser. No. 10/834,539 entitled “Extrusion Applicator Having Linear Motion Operability” filed on Apr. 29, 2004, and in U.S. application Ser. No. 10/834,503 entitled “Extrusion Applicator Having Rotational Operability” filed on Apr. 29, 2004.

Suitable apparatuses and methods for printing elastic elements in any orientation are described in U.S. application Ser. No. 10/811,671 entitled “Variable Stretch Composites and Methods of Making the Composite” filed on Mar. 29, 2004, and in U.S. application Ser. No. 10/811,527 entitled “Variable Stretch Composites and Methods of Making the Composite” filed on Mar. 29, 2004. For the printing of elastic strands, the individual elastic strands may be configured as lines or strands generally having widths less than about 2 mm and typically less than about 1 mm, in some embodiments. In some embodiments, linear elastic strands may be configured as bands generally having widths between about 2 mm and about 20 mm and aspect ratios ranging from about 2:1 to about 100:1. In some embodiments, the thickness of an elastic strand may be in the range of about 0.02 mm to about 5 mm and the basis weight is in the range of about 20 g/m² to about 300 g/m².

Referring again to FIG. 3, as discussed previously, the front side panels 124, the back side panels 130, and/or the intermediate side panels 33, utilized in the present invention may comprise laminated structures including a first substrate 310, 320 and a second substrate 314, 324 attached to an elastomeric element 312, 322 in some embodiments. The first substrate and/or the second substrate, in some embodiments, can be attached to the elastic element 312, 322 in a face to face orientation such that the elastomeric element is sandwiched between the first substrate and the second substrate.

The first and/or second substrates may comprise woven materials, nonwoven materials, combinations of woven and nonwoven materials, or laminated structures having woven or nonwoven materials. Suitable nonwoven materials for use in accordance with the present invention may comprise fibers made of polypropylene, polyethylene, polyester, nylon, cellulose, polyamide, or combinations of such materials. Fibers of one material or fibers of different materials or material combinations may be used in the nonwovens. Suitable processes for manufacturing nonwoven materials include spunbond, spunbond meltblown spunbond (SMS), spunbond meltblown meltblown spunbond (SMMS), carded and the like. Other suitable nonwoven materials include high elongation carded (HEC) nonwovens and deep activation polypropylene (DAPP) nonwovens. Any process known in the art may be used to make the nonwovens.

If a nonwoven is used, the nonwoven may comprise fibers that are bonded internally, including fibers that are needle punched, hydro entangled, spunbonded, thermally bonded, bonded by various types of chemical bonding such as latex bonding, powder bonding, and the like. The basis weight of the first nonwoven and/or second nonwoven may, for example, be in the range of about 10 gsm to about 100 gsm or any individual number within the range.

The present invention is further directed to a method of producing a disposable pant for wearing about the lower torso of a wearer, the method comprising the steps of joining a front side panel to the chassis in the first waist region, wherein the front side panel has a first stretchable width; joining a back side panel to the chassis in the second waist region, wherein the back side panel has a second stretchable width; joining the intermediate side panel to the front side panel, thereby creating a first fastening seam; joining the intermediate side panel to the back side panel, thereby creating a second fastening seam, wherein the first fastening seam is refastenable and the second fastening seam is non-refastenable; and positioning the first side panel and the second side panel to maintain a gap having a gap width of between about 1 percent to about 40 percent of the first stretchable width plus the second stretchable width. Optionally, the first fastening seam is created before the second fastening seam.

As discussed herein, the side panels can be joined to each other by any suitable method known in the art. Some examples of suitable joining methods include a variety of conventional bonding techniques including, but not limited to, adhesives, cohesives, thermal bonding, pressure bonding, mechanical bonding, ultrasonic bonding, fusion bonding, and/or any combination of any known methods of attaching such materials. Further, the first substrate, second substrate and the elastomeric element may be attached by any means of attachment known in the art. Some examples of suitable attaching means and/or methods for attaching include, but not limited to, adhesives, cohesives, thermal bonding, pressure bonding, mechanical bonding, ultrasonic bonding, fusion bonding, and/or any combination of any known methods of attaching such materials. In addition, the first side panel and the second side panel may be positioned to maintain a gap by any process known in the art for tracking and/or orienting webs to have the proper surfaces in contact with each other.

In some embodiments, the intermediate side panel 33 may comprise a single layer of material. For example, in some embodiments, the intermediate side panel 33 may comprise a nonwoven or a film. Examples of nonwovens include those discussed with regard to the first substrate and/or the second substrate. In some embodiments, the intermediate side panel 33 may comprise a material which is inextensible. Additionally, in some embodiments, the front side panels 124 and/or the second side panels 130 may comprise a single layer of material. Examples of suitable material are provided above.

Similar to the front side panels 124, back side panels 130, and intermediate side panels 33, the disposable absorbent article 100 of the present invention may comprise a variety of configurations utilizing a variety of materials. For example, in some embodiments, the topsheet 170 and the backsheet 174 can have length and width dimensions generally larger than those of the absorbent core 178. The topsheet 170 and the backsheet 174 can extend beyond the edges of the absorbent core 178, thereby forming the periphery of the disposable absorbent article 100. Additionally, the topsheet 170, the backsheet 174, and the absorbent core 178 may include many different materials and may be assembled in a variety of well known configurations, exemplary diaper materials and configurations are described generally in U.S. Pat. No. 3,860,003, U.S. Pat. No. 5,151,092, and U.S. Pat. No. 5,221,274.

Some examples of suitable topsheets are described further in U.S. Pat. No. 3,929,135; U.S. Pat. No. 4,324,246; U.S. Pat. No. 4,342,314; U.S. Pat. No. 4,463,045; U.S. Pat. No. 5,006,394; U.S. Pat. No. 4,609,518; U.S. Pat. No. 4,629,643. Any portion of the topsheet may be coated with a lotion as is known in the art. Examples of suitable lotions include those described in U.S. Pat. No. 5,607,760; U.S. Pat. No. 5,609,587; U.S. Pat. No. 5,635,191; U.S. Pat. No. 5,643,588; U.S. Pat. No. 5,968,025; U.S. Pat. No. 6,716,441; and PCT Publication No. WO 95/24173.

Further, the topsheet 170 may be fully or partially elasticated or may be foreshortened so as to provide a void space between the topsheet and the absorbent core. Exemplary structures including elasticized or foreshortened topsheets are described in more detail in U.S. Pat. No. 4,892,536; U.S. Pat. No. 4,990,147; U.S. Pat. No. 5,037,416; and U.S. Pat. No. 5,269,775.

An example of a suitable backsheet 174 for use in the disposable absorbent article of the present invention may be impervious to liquids (e.g., urine) and comprise a thin plastic film such as a thermoplastic film having a thickness, for example, of about 0.012 mm (0.5 mil) to about 0.051 mm (2.0 mils). Suitable backsheet films include those manufactured by Tredegar Corporation, based in Richmond, Va., and sold under the trade name CPC2 film. Other suitable backsheet materials may include breathable materials which permit vapors to escape from the pull-on garment while still preventing exudates from passing through the backsheet. Suitable breathable materials may include materials such as woven webs, nonwoven webs, composite materials such as film-coated nonwoven webs, microporous films such as manufactured by Mitsui Toatsu Co., of Japan under the designation ESPOIR NO and by Tredegar Corporation of Richmond, Va. and sold under the designation EXAIRE, and monolithic films such as manufactured by Clopay Corporation, Cincinnati, Ohio under the name HYTREL blend P18-3097. Some breathable composite materials are described in greater detail in PCT Application No. WO 95/16746; U.S. Pat. No. 5,938,648; U.S. Pat. No. 5,865,823; and U.S. Pat. No. 5,571,096.

The backsheet 174, or any portion thereof, may be elastically extensible in one or more directions. In one embodiment, the backsheet may comprise a structural elastic-like film (“SELF”) web. A structural elastic-like film web is an extensible material that exhibits an elastic-like behavior in the direction of elongation without the use of added elastic materials and is described in more detail in U.S. Pat. No. 5,518,801. In alternate embodiments, the backsheet 174 may comprise elastic films, foams, strands, or combinations of these or other suitable materials with nonwovens or synthetic films.

A suitable absorbent core 178 for use in the present invention may comprise any absorbent material which is generally compressible, conformable, non-irritating to the wearer's skin, and capable of absorbing and retaining liquids such as urine and other certain body exudates. In addition, the configuration and construction of the absorbent core may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). Suitable absorbent structures for use as the absorbent core 178 are described in U.S. Pat. No. 4,610,678; U.S. Pat. No. 4,673,402; U.S. Pat. No. 4,834,735; U.S. Pat. No. 4,888,231; U.S. Pat. No. 5,137,537; U.S. Pat. No. 5,147,345; U.S. Pat. No. 5,342,338; U.S. Pat. No. 5,260,345; U.S. Pat. No. 5,387,207; and U.S. Pat. No. 5,625,222.

The backsheet 174 may be attached to the topsheet 170, the absorbent core 178, or any other element of the disposable absorbent article 100 by any attachment means known in the art. For example, the attachment means may include a uniform continuous layer of adhesive, a patterned layer of adhesive, or an array of separate lines, spirals, or spots of adhesive. Some suitable attachment means are disclosed in U.S. Pat. No. 4,573,986; U.S. Pat. No. 3,911,173; U.S. Pat. No. 4,785,996; and U.S. Pat. No. 4,842,666. Examples of suitable adhesives are manufactured by H. B. Fuller Company of St. Paul, Minn. and marketed as HL-1620 and HL-1358-XZP. Alternatively, the attachment means may comprise heat bonds, pressure bonds, ultrasonic bonds, dynamic mechanical bonds, or any other suitable attachment means or combinations of these attachment means as are known in the art.

Various sublayers may be disposed between the topsheet 170 and the backsheet 174. The sublayer may be any material or structure capable of accepting, storing or immobilizing bodily exudates. Thus, the sublayer may include a single material or a number of materials operatively associated with each other. Further, the sublayer may be integral with another element of the pull-on garment or may be one or more separate elements attached directly or indirectly with one or more elements of the disposable absorbent article. Further, the sublayer may include a structure that is separate from the absorbent core or may include or be part of at least a portion of the absorbent core.

Suitable materials for use as the sublayer may include large cell open foams, macro-porous compression resistant nonwoven highlofts, large size particulate forms of open and closed cell foams (macro and/or microporous), highloft nonwovens, polyolefin, polystyrene, polyurethane foams or particles, structures comprising a multiplicity of vertically oriented looped strands of fibers, absorbent core structures described above having punched holes or depressions, and the like. (As used herein, the term “microporous” refers to materials which are capable of transporting fluids by capillary action. The term “macroporous” refers to materials having pores too large to effect capillary transport of fluid, generally having pores greater than about 0.5 mm in diameter and, more specifically, having pores greater than about 1.0 mm in diameter.) One embodiment of a sublayer includes a mechanical fastening loop landing element, having an uncompressed thickness of about 1.5 millimeters available as XPL-7124 from the 3M Corporation of Minneapolis, Minn. Another embodiment includes a 6 denier, crimped and resin-bonded nonwoven highloft having a basis weight of 110 grams per square meter and an uncompressed thickness of 7.9 millimeters which is available from the Glit Company of Wrens, Ga. Other suitable absorbent and nonabsorbent sublayers are described in U.S. Pat. No. 6,680,422 and U.S. Pat. No. 5,941,864. Further, the sublayer, or any portion thereof, may include or be coated with a lotion or other known substances to add, enhance or change the performance or other characteristics of the element.

Embodiments of the present invention may include acquisition layers and dusting layers, each of which are well known in the art. Acquisition layer are further discussed in U.S. Pat. No. 5,460,622. Dusting layers are further discussed in U.S. Pat. No. 4,888,231.

Embodiments of the present invention may also include pockets for receiving and containing waste, spacers which provide voids for waste, barriers for limiting the movement of waste in the article, compartments or voids which accept and contain waste materials deposited in the pull-on garment, and the like, or any combinations thereof. Examples of pockets and spacers for use in absorbent products are described in U.S. Pat. No. 5,514,121; U.S. Pat. No. 5,171,236; U.S. Pat. No. 5,397,318; U.S. Pat. No. 5,540,671; U.S. Pat. No. 6,168,584; U.S. Pat. No. 5,306,266; and U.S. Pat. No. 5,997,520. Examples of compartments or voids in an absorbent article are disclosed in U.S. Pat. No. 4,968,312; U.S. Pat. No. 4,990,147; U.S. Pat. No. 5,062,840; and U.S. Pat. No. 5,269,755. Examples of suitable transverse barriers are described in U.S. Pat. No. 5,554,142; PCT Patent WO 94/14395; and U.S. Pat. No. 5,653,703. Examples of other structures suitable for management of low viscosity feces are disclosed in U.S. Pat. No. 5,941,864; U.S. Pat. No. 5,977,430; and U.S. Pat. No. 6,013,063.

The front side panel 124, back side panel 130, and intermediate side panel 33, collectively, “panels”, of the present invention may be incorporated into a variety of consumer and commercial goods that may benefit from having a refastenable seam and a non-refastenable seam. For example, the panels may be included in surgical garments, surgical masks, wound wraps, bandages, body wraps, catemenials, and disposable absorbent articles, e.g. diapers, pull-on diapers, bibs, etc.

Test Methods:

Method for Measurement of Gap Width, First Stretchable Width, Second Stretchable Width, and Widths of the Fastening Seams:

All testing is to occur in conditions controlled to 22° C.±2° C., 50% Relative Humidity±10% Relative Humidity. Samples are conditioned at these conditions at least 24 hours prior to testing. All distance measures made to the nearest 0.5 mm, using a calibrated ruler, calipers, or image analysis system. All cuts are to be made with either scissors or razor knives.
1. Select a representative product. If the fastening seams are prefastened, do not unfasten them.
2. Cut the product approximately parallel to lateral centerline 60 at a location near the lateral centerline.
3. Mark a location on each side panel to indicate the longitudinal direction of the article.
4. Cut the side panels off the product by cutting the product at a location laterally inboard of the innermost edges of each side panel. The cut is to be made as close to the innermost edges of each side panel as possible without cutting any side panel material.
5. Cut away excess material (i.e., leg cuff, topsheet, backsheet, etc.) remaining at the lower edge of the side panel. The cut is to be made as close to the lower edges of each side panel as possible without cutting any side panel material.
6. Lay the assembled first side panel, second side panel, and intermediate side panels on a flat surface. The sample should be flat and generally free of wrinkles, other than inherent wrinkles in the materials (i.e., corrugations in stretch materials, etc.). Note: if the product was not prefastened at 1 or more refastenable seams, fasten the seam. If fastening location can vary (e.g., is adjustable), measures are to be completed for samples at each extreme of fastening location possible.
7. Identify the key edges as defined within the specification.
8. Measure the first stretchable width, second stretchable width, and gap width in a direction perpendicular to the longitudinal direction of the article—as defined in the specification.
9. Calculations:
   a. Gap Width as % of first and second stretchable width=100*{gap width/(first stretchable width+second stretchable width)}
   b. Gap Width as % of first stretchable width=100*(gap width/first stretchable width)
   c. Gap Width as % of second stretchable width=100*(gap width/second stretchable width)

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article for wearing about the lower torso of a wearer, the disposable absorbent article including a chassis having a topsheet, a backsheet attached to at least a portion of the topsheet, an absorbent core disposed between the topsheet and the backsheet; a first waist region disposed adjacent to a first waist edge, a second waist region disposed adjacent to a second waist edge, and a crotch region disposed between the first waist region and the second waist region; a first longitudinal edge and a second longitudinal edge, the disposable absorbent article further comprising:
a first side panel extending outward from the first longitudinal edge, wherein the first side panel is disposed in the first waist region, wherein the first side panel comprises a first fastening element, and wherein the first side panel has a first stretchable width;
a second side panel extending outward from the first longitudinal edge, wherein the second side panel is disposed in the second waist region, and wherein the second side panel has a second stretchable width;
an intermediate side panel joined to the second panel thereby forming a non-refastenable seam, wherein the intermediate side panel includes a second fastening element capable of engaging the first fastening element thereby forming a refastenable seam; and
a gap disposed between the first side panel and the second side panel, the gap having a gap width, wherein the gap width is between about 1 percent to about 40 percent of the first stretchable width plus the second stretchable width.

2. The disposable absorbent article of claim 1 wherein the gap width is between about 3 percent to about 20 percent of the first stretchable width plus the second stretchable width.

3. The disposable absorbent article of claim 1 wherein the gap width is between about 5 percent to about 10 percent of the first stretchable width plus the second stretchable width.

4. The disposable absorbent article of claim 1 wherein the gap width is less than about 60 percent of the first stretchable width.

5. The disposable absorbent article of claim 1 wherein the gap width is less than about 45 percent of the first stretchable width.

6. The disposable absorbent article of claim 1 wherein the gap width is less than about 30 percent of the first stretchable width.

7. The disposable absorbent article of claim 1 wherein the gap width is less than about 50 percent of the second stretchable width.

8. The disposable absorbent article of claim 1 wherein the gap width is less than about 35 percent of the second stretchable width.

9. The disposable absorbent article of claim 1 wherein the gap width is less than about 30 percent of the second stretchable width.

10. A disposable pant for wearing about the lower torso of a wearer, the disposable pant including a chassis having a topsheet, a backsheet attached to at least a portion of the topsheet, an absorbent core disposed between the topsheet and the backsheet; a first waist region disposed adjacent to a first waist edge, a second waist region disposed adjacent to a second waist edge, and a crotch region disposed between the first waist region and the second waist region; a first longitudinal edge and a second longitudinal edge; and a waist opening and a pair of leg openings; the disposable pant further comprising:
a pair of front side panels each having a first stretchable width and each having an outermost edge, a first front side panel extending outward from the first longitudinal edge in the front waist region and a second front side panel extending outward from the second longitudinal edge in the front waist region;
a pair of back side panels each having a second stretchable width and each having an outermost edge, a first back side panel extending outward from the first longitudinal edge in the back waist region and a second back side panel extending outward from the second longitudinal edge in the back waist region;
a pair of intermediate side panels, one of the pair of intermediate side panels being joined to the first front side panel and the first back side panel thereby forming a first non-refastenable seam and a first refastenable seam, and one of the pair of intermediate side panels being joined to the second front side panel and the second back side panel thereby forming a second non-refastenable seam and a second refastenable seam;
a first gap disposed between the first front side panel and the first back side panel, the first gap having a first gap width; and
a second gap disposed between the second front side panel and the second back side panel, the second gap having a second gap width,
wherein the first gap width and the second gap width are each between about 1 percent to about 40 percent of the first stretchable width plus the second stretchable width of their respective front side panels and back side panels.

11. The disposable absorbent article of claim 10 wherein the first gap width and the second gap width are between about 3 percent to about 20 percent of the first stretchable width plus the second stretchable width of their respective front and back side panels.

12. The disposable absorbent article of claim 11 wherein the first gap width and the second gap width are between about 5 percent to about 10 percent of the first stretchable width plus the second stretchable width of their respective front and back side panels.

13. The disposable absorbent article of claim 11 wherein the first gap width and the second gap width are less than about 60 percent of the first stretchable width of the first front side panel and the second front side panel, respectively.

14. The disposable absorbent article of claim 11 wherein the first gap width and the second gap width are less than about 45 percent of the first stretchable width of the first front side panel and the second front side panel, respectively.

15. The disposable absorbent article of claim 11 wherein the first gap width and the second gap width are less than about 30 percent of the first stretchable width of the first front side panel and the second front side panel, respectively.

16. The disposable absorbent article of claim 11 wherein the first gap width and the second gap width are less than about 50 percent of the second stretchable width of the first back side panel and the second back side panel, respectively.

17. The disposable absorbent article of claim 11 wherein the first gap width and the second gap width are less than about 35 percent of the second stretchable width of the first back side panel and the second back side panel, respectively.

18. The disposable absorbent article of claim 11 wherein the first gap width and the second gap width are less than about 20 percent of the second stretchable width of the first back side panel and the second back side panel, respectively.

19. A method of producing a disposable pant for wearing about the lower torso of a wearer, the disposable pant including a chassis having a topsheet, a backsheet attached to at least a portion of the topsheet, an absorbent core disposed between the topsheet and the backsheet; a first waist region disposed adjacent to a first waist edge, a second waist region disposed adjacent to a second waist edge, and a crotch region disposed between the first waist region and the second waist region; a first longitudinal edge and a second longitudinal edge; and a waist opening and a pair of leg openings; the method comprising the steps of:
joining a front side panel to the chassis in the first waist region, wherein the front side panel has a first stretchable width;
joining a back side panel to the chassis in the second waist region, wherein the back side panel has a second stretchable width;
joining the intermediate side panel to the front side panel, thereby creating a first fastening seam;
joining the intermediate side panel to the back side panel, thereby creating a second fastening seam, wherein the first fastening seam is refastenable and the second fastening seam is non-refastenable; and
positioning the first side panel and the second side panel to maintain a gap having a gap width of between about 1 percent to about 40 percent of the first stretchable width plus the second stretchable width.

20. The method of claim 19 wherein the first fastening seam is created before the second fastening seam.

21. A package comprising a plurality of disposable absorbent articles for wearing about the lower torso of a wearer, each of the disposable absorbent articles including a chassis having a topsheet, a backsheet attached to at least a portion of the topsheet, an absorbent core disposed between the topsheet and the backsheet; a first waist region disposed adjacent to a first waist edge, a second waist region disposed adjacent to a second waist edge, and a crotch region disposed between the first waist region and the second waist region; a first longitudinal edge and a second longitudinal edge, the disposable absorbent article further comprising:
a first side panel extending outward from the first longitudinal edge, wherein the first side panel is disposed in the first waist region, wherein the first side panel comprises a first fastening element, and wherein the first side panel has a first stretchable width;
a second side panel extending outward from the first longitudinal edge, wherein the second side panel is disposed in the second waist region, and wherein the second side panel has a second stretchable width;
an intermediate side panel joined to the second panel thereby forming a non-refastenable seam, wherein the intermediate side panel includes a second fastening element capable of engaging the first fastening element thereby forming a refastenable seam; and
a gap disposed between the first side panel and the second side panel, the gap having a gap width, wherein the gap width is between about 1 percent to about 40 percent of the first stretchable width plus the second stretchable width,
wherein each of the plurality of disposable absorbent articles prefastened in the package.

22. A package comprising a plurality of disposable pants for wearing about the lower torso of a wearer, each of the disposable pants including a chassis having a topsheet, a backsheet attached to at least a portion of the topsheet, an absorbent core disposed between the topsheet and the backsheet; a first waist region disposed adjacent to a first waist edge, a second waist region disposed adjacent to a second waist edge, and a crotch region disposed between the first waist region and the second waist region; a first longitudinal edge and a second longitudinal edge; and a waist opening and a pair of leg openings; the disposable pant further comprising:
a pair of front side panels each having a first stretchable width and each having an outermost edge, a first front side panel extending outward from the first longitudinal edge in the front waist region and a second front side panel extending outward from the second longitudinal edge in the front waist region;
a pair of back side panels each having a second stretchable width and each having an outermost edge, a first back side panel extending outward from the first longitudinal edge in the back waist region and a second back side panel extending outward from the second longitudinal edge in the back waist region;
a pair of intermediate side panels, one of the pair of intermediate side panels being joined to the first front side panel and the first back side panel thereby forming a first non-refastenable seam and a first refastenable seam, and one of the pair of intermediate side panels being joined to the second front side panel and the second back side panel thereby forming a second non-refastenable seam and a second refastenable seam;
a first gap disposed between the first front side panel and the first back side panel, the first gap having a first gap width; and
a second gap disposed between the second front side panel and the second back side panel, the second gap having a second gap width,
wherein the first gap width and the second gap width are each between about 1 percent to about 40 percent of the first stretchable width plus the second stretchable width of their respective front side panels and back side panels, and wherein each of the plurality of pants is prefastened in the package.

23. A disposable absorbent article for wearing about the lower torso of a wearer, the disposable absorbent article including a chassis having a topsheet, a backsheet attached to at least a portion of the topsheet, an absorbent core disposed between the topsheet and the backsheet; a first waist region disposed adjacent to a first waist edge, a second waist region disposed adjacent to a second waist edge, and a crotch region disposed between the first waist region and the second waist region; a first longitudinal edge and a second longitudinal edge, the disposable absorbent article further comprising:
a first side panel extending outward from the first longitudinal edge, wherein the first side panel is disposed in the first waist region, wherein the first side panel comprises a first fastening element, and wherein the first side panel has a first stretchable width;
a second side panel extending outward from the first longitudinal edge, wherein the second side panel is disposed in the second waist region, and wherein the second side panel has a second stretchable width;
an intermediate side panel joined to the first side panel via first configuration bonds, wherein the intermediate side panel includes a second fastening element capable of engaging the first fastening element thereby forming a refastenable seam, wherein said first orientation and second orientation are different, and wherein the intermediate side panel is joined to the second side panel thereby forming a non-refastenable seam; and
a gap disposed between the first side panel and the second side panel, the gap having a gap width, wherein the gap width is between about 1 percent to about 40 percent of the first stretchable width plus the second stretchable width, wherein the refastenable seam is prefastened in a first orientation,
